(19) United States
(12) Patent Application Publication
(10) Pub. No.: US 2026/0031237 A1
(43) Pub. Date: Jan. 29, 2026

(54) Heart Murmur Visualization and Analysis

(71) Applicant: Scott Pless, Las Vegas, NV (US)

(72) Inventor: Scott Pless, Las Vegas, NV (US)

(21) Appl. No.: 18/783,715

(22) Filed: Jul. 25, 2024

(51) Int. Cl. G16H 50/50

(52) U.S. Cl. CPC G16H 50/50

(57) ABSTRACT

Heart murmur methods and analyzers are provided with real-time, colorized, three-dimensional, visual spectrograms of hearts and heart murmurs in clinical and/or surgical settings of animal and human patients.

### FIELD OF THE INVENTION

The invention relates to the visualization and analysis of heart murmurs.

### BACKGROUND OF THE INVENTION

Within the field of veterinary and medical physical examination diagnostics, practitioners face a lack of an accurate and affordable method of information collection when it comes to gathering diagnostic data to objectively detect, evaluate, track, and analyze clinical and subclinical severity, identification, same species comparison, cross species comparison, approximate valve localization, exact anatomical localized triangulation, and progression of heart murmurs in a real-time, visual, colorized, three-dimensional acoustic spectrum in a clinical and/or surgical setting of patients within the applications of the field of veterinary and human medicine.

Improved methods for detection, evaluation, tracking, and analysis of heart murmurs in patients is needed. Such methods would provide more accuracy and more uniform, more available, and easier tracking and comparative and progression-detection abilities, be more affordable, and have other advantages over current methods.

### SUMMARY OF THE INVENTION

This invention includes methods of providing practitioners real-time, visual, colorized, three-dimensional, acoustic spectrum models of auscultatory physical exam diagnostic information used for evaluation of heart murmurs. Audio data is collected from heart murmurs that is then rendered into visual, colorized, three-dimensional spectrograms for further analysis.

Principal objects of certain embodiments of this invention are to provide accurate and detailed acoustic mapping to a real-time three-dimensional acoustic spectrum of heart murmurs for use in diagnosis, monitoring, or surgical operation. Additional objects of certain embodiments of this invention are to aid practitioners during procedures by providing them with real-time, three-dimensional, acoustic spectrum information of heart murmurs whilst the patients are being operated on as it helps guide the course of the surgery and serves as a predictive indicator of the surgical success and/or failure. Further objects of certain embodiments of this invention are to track subclinical and clinical three-dimensional acoustic spectrum mapped acoustic changes of heart murmurs to increase descriptive information to further the ability to detect, evaluate, and analyze clinical and subclinical severity, identification, same species comparison, cross species comparison, approximate valve localization, exact anatomical localized triangulation, and progression of heart murmurs of patients.

In particularly preferred embodiments, methods of analyzing a heart of a patient for a heart murmur are provided. The methods comprise (a) capturing a digital audio file of the heart; (b) rendering the digital audio file into a visual, colorized, three-dimensional spectrogram; (c) inputting the spectrogram into an image recognition artificial intelligence model trained by previously acquired and graded spectrograms of the same and/or different hearts; and (c) outputting the visual, colorized, three-dimensional spectrogram of the heart and an analysis of the spectrogram of the heart by the artificial intelligence model, the analysis comprising the identification of any heart murmur.

In other particularly preferred embodiments, heart murmur analyzers for analyzing a heart of a patient for a heart murmur are provided. The analyzers comprise: (a) data interface for capturing a digital audio file of the heart; (b) a memory for storing the digital audio file of the heart, and the memory also storing computer executable instructions, wherein the computer executable instructions comprise instructions for (i) rendering digital audio files of hearts into visual, colorized, three-dimensional spectrograms of hearts, and (ii) an image recognition artificial intelligence model that analyzes spectrograms of hearts; (c) a computer and processor for processing the computer executable instructions, the processing comprising (i) rendering the digital audio file into a visual, colorized, three-dimensional spectrogram and (ii) inputting the spectrogram into the artificial intelligence model to obtain an analysis of the spectrogram from the artificial intelligence model; and (d) a display for outputting information, the information comprising the spectrogram of the heart of interest and the analysis of the spectrogram from the artificial intelligence model, wherein the analysis comprises an identification of any heart murmur.

Certain preferred embodiments of this invention provide the practitioner with a number of possible advantages, including the ability to objectively detect, evaluate, and analyze clinical and subclinical severity, identification of heart murmurs and associated anomalies, same species comparisons, cross species comparisons, approximate valve localization, exact anatomical localized triangulation, and progression of heart murmurs, in a real-time, visual, colorized, three-dimensional acoustic spectrum by using the degree of acoustic fluctuations as they are mapped to the real-time, visual, colorized, three-dimensional acoustic spectrum model of the heart murmurs of patients within the applications of the field of veterinary and human medicine.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIG. 1 shows a gray-scale depiction of an embodiment of this invention that is three visual, colorized, three-dimensional spectrograms that were each rendered from a digital audio file of three different hearts at different times.

### DETAILED DESCRIPTION OF THE INVENTION

The invention provides for the visualization of real-time, three-dimensional, acoustic spectrum models of heart murmurs. In certain preferred embodiments of this invention, a real-time, visual, colorized, three-dimensional topographic audio scan of heart murmurs in the form of a rendered spectrogram is provided by using the degree of audio fluctuations detected or recorded by a digital audio recorder. Image recognition artificial intelligence models are used that are trained on the renderings of multiple graded digital audio files to provide analysis of the heart and heart murmurs and comparative information to practitioners, patients, and others.

These preferred embodiments may include obtaining auscultatory physical exam diagnostic information for diagnostic evaluation of heart murmurs. This may specifically include (a) the identification of heart murmurs, (b) the evaluation of the severity grading of heart murmurs, (c) the evaluation of the progression of heart murmurs, (d) the evaluation of the predictive estimation of heartbeat cessation, (e) the evaluation of the auscultatory peripheral blood flow turbulence sounds, (f) the evaluation of the same patient comparison of heart murmurs, (g) the evaluation of the same species comparison of heart murmurs, (h) the evaluation of the cross species comparison of heart murmurs, (i) the evaluation of the approximate valve localization of heart murmurs, and/or (j) the evaluation of the exact anatomical localized triangulation of heart murmurs. Additional uses and advantages of the invention are described herein and/or will be apparent to a person of skill in the art.

For certain preferred embodiments, this may also include the training of image recognition artificial intelligence from renderings of multiple graded digital audio files to provide the capability for (a) the identification of heart murmurs, (b) the evaluation of the severity grading of heart murmurs, (c) the evaluation of the progression of heart murmurs, (d) the evaluation of the predictive estimation of heartbeat cessation, (e) the evaluation of the auscultatory peripheral blood flow turbulence sounds, (f) the evaluation of the same patient comparison of heart murmurs, (g) the evaluation of the same species comparison of heart murmur, (h) the evaluation of the cross species comparison of heart murmurs, (i) the evaluation of the approximate valve localization of heart murmurs, and/or (j) the evaluation of the exact anatomical localized triangulation of heart murmurs. Additional uses of artificial intelligence trained with embodiments of this invention are described herein and/or will be apparent to a person of skill in the art.

The subject matter of this disclosure is now described with reference to the following examples. These examples are provided for the purpose of illustration only, and the subject matter is not limited to these examples, but rather encompasses all variations which are evident as a result of the teaching provided herein.

### Example 1

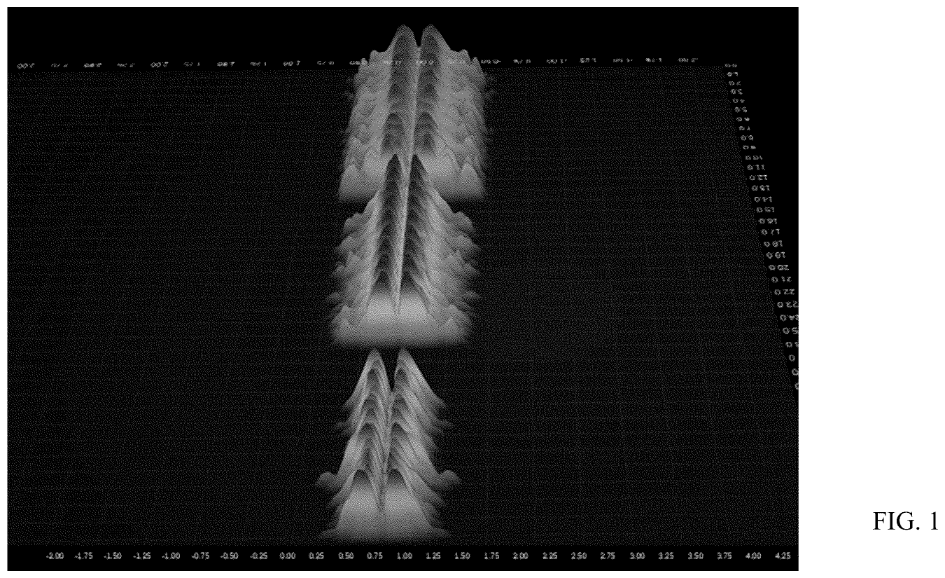

FIG. 1 shows an embodiment of this invention. It is a depiction of a visual, colorized, three-dimensional spectrogram of three digital audio files. It is, however, in gray scale in the published version. The spectrogram resulted from the plotting of digital audio files where the axis for X, Y, and Z, are Time, Frequency, and Power.

While FIG. 1 is shown in gray-scale, the preferred embodiments are colorized. For example, FIG. 1 shows a series of peaks. In the colorized spectrogram of this invention, the top of the peaks may be red and the bottom of the rendering is blue, with a continuous change of color from blue to green to yellow to orange to red, when looking from bottom to top.

FIG. 1 shows three separate spectrograms from three different patients. The lowest spectrogram is from a cat with a normal heart, the middle spectrogram is a grade 3 heart murmur from a cat, and the top spectrogram is a grade 5 heart murmur from a cat. The grade 5 patient died from heart failure shortly after the spectrogram was made.

### Example 2

A digital audio recorder with the capability to pick-up and record heart sounds in a patient is used in this embodiment of the invention. In some embodiments, this digital audio recorder may comprise a device that captures the digital audio output of a digital stethoscope.

The digital audio recorder is placed on the chest of the patient at the point of maximal intensity, where the heart murmur is heard the loudest.

The digital audio recorder is used to record a digital audio file of the heart murmur, including at the point of maximal intensity.

In some embodiments, the location of a murmur can be found using triangulation from multiple recorders or recordings in a virtual three-dimensional space of the chest. Multiple recordings of different patients, multiple recordings of the same patient marked over time with percentage worsening or improvement (e.g., by the artificial intelligence model), and cross species comparisons (e.g., with logic rules in place to allow for the comparison of different species with different sized chest areas and/or different heart rates), can also be determined in certain embodiments.

Each digital audio file is transferred to a digital computing device, most preferably a mobile digital computing device that is handheld.

Each digital audio file is rendered and colorized by the digital computing device into a three-dimensional heart colorized spectrogram showing three-dimensional morphology of the heart.

The three-dimensional morphology rendering (e.g., without sound) is analyzed by an image recognition artificial intelligence model and in preferred embodiments of this example a heart murmur grade is given by the artificial intelligence model based upon the artificial intelligence being trained on multiple previously recorded heart murmur renderings of digital audio files with heart murmur grading.

Preferred artificial intelligence image recognition uses machine-based identification and categorization of objects within images. These capabilities use artificial intelligence to emulate human-level visual understanding. Examples of such algorithms include YOLOv9, Faster RCNN, etc.

The artificial intelligence can also be applied to multiple previously recorded heart murmur renderings of digital audio files with the comparing of them to each other and any new test renderings of digital audio files and with the determination of any heart murmur changes (e.g., sub-clinical, improvement/deterioration detection) or other useful comparative information.

The heart murmur rendering can also be compared to renderings of other patients for comparative information used to confirm diagnosis, establish a prognosis, etc.

Heart murmur grades can be communicated to clients, patients, and other practitioner and personnel (e.g., medical, veterinary) without the need of audio capabilities due to the three-dimensional colorized rendering of embodiments of this invention.

The artificial intelligence can be trained further in some embodiments by the input of daily use data, most preferably curated by practitioners comprised of an internal team.

These embodiments are beneficial to companion animal veterinary medicine, large animal medicine, livestock, exotic animal medicine, and human medicine.

It is notable that there is a current lack of sensitivity in detecting subclinical heart murmurs and subtle heart murmur changes either worsening or improving, as well as heart murmur grading in general. Intra-grade changes are not currently recorded due to a lack of technology such as used in methods of this invention.

Certain of these methods allow for a quantitative improvement or worsening percentage to be given by the artificial intelligence to help inform the practitioner if the treatment is working or not and to what extent over time.

Certain of these methods also provide for the first way for hearing impaired clients and medical personnel to effectively see and evaluate heart murmurs, which poses a great challenge in medicine and veterinary practice today.

It is believed that methods of this invention may be applied to help save millions of lives as heart disease is one of the leading causes of death in animals and humans. These methods also permit cross species comparisons of heart murmurs for the first time. In certain embodiments, these can also be displayed as a three dimensional colorized hologram as well as be incorporated into additional and/or general medical artificial intelligence applications.

### Example 3

The artificial intelligence of certain embodiments of this invention can be trained on the renderings of digital audio files of this invention that are described herein. The methods of training may comprise receiving and processing by a digital computer device of renderings of multiple digital audio files of heart murmurs that have been graded and otherwise evaluated (preferably by a team of trained personnel), generating a model of such, and inputting a test rendering of a digital audio file into the trained artificial intelligence model to obtain information concerning the test rendering of the digital audio file. The utility of the artificial intelligence model described herein will generally be increased by including as much information in the grading of the training renderings of the digital audio files as practicable.

The artificial intelligence is contained in or accessible to a digital computing device (e.g., desktop computer, laptop computer, tablet computer, mobile handheld devices, mobile telephone, computer terminal or its equivalent) or is otherwise accessible on a server and/or network (e.g., cloud). It may be separate or part of the digital audio recording device used to create the digital audio file. A person skilled in the art will understand and have knowledge as to the different ways computing devices and digital audio recorders can be applied in such a manner after reading this specification.

The rendering of the digital audio file, which is preferably colorized, is done by computer executable instructions (e.g., hardware and software) contained or accessible to the digital computing device into a three-dimensional heart murmur visual rendering showing three-dimensional morphology of the heart. A person of skill in the art knows of the available audio visualizer hardware and software that can be used or modified and used (e.g., Filmora Audio Visualizer, audiograms, waveforms) or that can be applied to affect the objects of this invention and generate a useful visual rendering of a heart and a heart with a heart murmur.

This three-dimensional morphology visual rendering (e.g., without sound) is preferably available to be shared with clients, patients and/or other practitioners electronically and/or visually (e.g., displayed on a monitor or other screen). In certain of the most preferred embodiments, the visual rendering can be observed in real time (i.e., within a very short period of time after digital processing) by the practitioners and/or others as a digital audio recording device is passed over and/or held over the heart.

In a particularly preferred embodiment, the artificial intelligence model is trained by three-dimensional color image recognition alone of the waveform from the digital audio file and not audio processing. The artificial intelligence model will have these three-dimensional, colorized, spectrograms tagged by a qualified doctor in a database for which the artificial intelligence model will be trained on thousands of tagged heart murmur spectrograms. The artificial intelligence model will continually learn from each new entry. The artificial intelligence model output would just assign each of the waveforms a class of murmur with an internal confidence score attached to the grade determined by the waveform image recognition by the artificial intelligence model. The artificial intelligence model will also have tagging for artifacts and anomalies such as if a cat is purring or if the doctor bumps the stethoscope, which will allow the artificial intelligence to identify such artifacts and anomalies.

The artificial intelligence model in certain embodiments will place a large number next to the waveform determining the grade along with displaying a smaller number of the percent change from the last murmur recording from that patient or a murmur it is being compared to. For example, it may show grade 3 murmur with a 15% worsening from the last recording 6 months ago, indicating that the heart disease is worsening sub-clinically. And then 6 months further into the future the murmur may be recorded again and be graded again as a 3 but showing it has improved by 5% due to the heart medication being taken regularly.

### Example 4

In this example, a heart murmur analyzer device comprised of multiple components is provided for analysis of heart murmurs. The analyzer comprises (a) a display (e.g., touchscreen, monitor and keyboard) for observing (and operating controls in some embodiments) and visual renderings of digital audio files, (b) a computer and processor for processing computer executable instructions, (c) memory for storing computer executable instructions (e.g., instructions for artificial intelligence models, for rendering a digital audio file into a visual, colorized, three-dimensional spectrogram) and digital audio files from patients, and (d) a data interface for capturing of digital audio files (e.g., directly or transferred from another device). Other components may also be used in or with the analyzer.

The analyzer is used in a method for analysis of a heart murmur by capturing at least one digital audio file collected from the heart of a patient, rendering the digital audio file into at least one visual, colorized, three-dimensional spectrogram, inputting the spectrogram into at least one artificial intelligence model trained on multiple spectrograms from training files (e.g., previously graded visual, colorized, three-dimensional spectrograms), and outputting an analysis of the heart murmur that comprises a spectrogram of the heart murmur. Additional actions may also be used in the method for analysis.

The analyzer can also be comprised as multiple modules included in one discrete unit or spread out over several separate but connected and/or connectable units. These modules may comprise (a) an output module (e.g., a display with hardware and software for a touchscreen, a printer, a projector, etc.) for observing information such as controls for the analyzer and visual renderings (visual, colorized, three-dimensional spectrograms) of digital audio files, (b) a computer and processor module for processing computer executable instructions, (c) a memory module for storing computer executable instructions (e.g., instructions for artificial intelligence models, for rendering a digital audio file into a visual colorized, three-dimensional spectrogram) and digital audio files from patients, and (d) a data interface module for capturing of digital audio files (e.g., directly or transferred from another device). Other components/modules may also be used in or with the analyzer.

The analyzer and method can offer a more advantageous, accurate, less expensive, more reproduceable heart murmur analysis, including original diagnosis, prognosis, comparisons, and improvement/deterioration tracking than any devices or methods used previously.

### OTHER EMBODIMENTS

Although the present invention has been described with reference to teaching, examples and preferred embodiments, one skilled in the art can easily ascertain its essential characteristics, and without departing from the spirit and scope thereof can make various changes and modifications of the invention to adapt it to various usages and conditions. Those skilled in the art will recognize or be able to ascertain using no more than routine experimentation, many equivalents to the specific embodiments of the invention described herein. Such equivalents are encompassed by the scope of the present invention.

What is claimed is:

1. A method of analyzing a heart of a patient for a heart murmur, the method comprising;
a. capturing a digital audio file of the heart;
b. rendering the digital audio file into a first spectrogram, wherein the first spectrogram is a visual, colorized, three-dimensional spectrogram and the X, Y, and Z axis of the spectrogram are Time, Frequency, and Power;
c. inputting the first spectrogram into an artificial intelligence model trained by previously acquired and graded multiple spectrograms of the same and/or different hearts; and
d. outputting the first spectrogram and an analysis of the first spectrogram by the artificial intelligence model, said analysis comprising the identification of any heart murmur.

2. The method of claim 1, wherein the outputting of the first spectrogram and the analysis of the first spectrogram is provided in real-time.

3. The method of claim 1, wherein the analysis further comprises an evaluation of the severity grading of any heart murmur.

4. The method of claim 1, wherein the analysis further comprises an evaluation of the progression of any heart murmur.

5. The method of claim 1, wherein the analysis further comprises an evaluation of the predictive estimation of heartbeat cessation.

6. The method of claim 1, wherein the analysis further comprises an evaluation of the auscultatory peripheral blood flow turbulence sounds.

7. The method of claim 1, wherein the analysis further comprises an evaluation of multiple spectrograms from the same patient from one or more previous examinations of the patient.

8. The method of claim 1, wherein the analysis further comprises comparisons of multiple spectrograms from the same species as the patient with the first spectrogram from the patient.

9. The method of claim 1, wherein the analysis further comprises an evaluation of multiple spectrograms across different species compared with the first spectrogram from the patient.

10. The method of claim 1, wherein the analysis further comprises an evaluation of the approximate valve localization of any heart murmur.

11. The method of claim 1, wherein the analysis further comprises an evaluation of the exact anatomical localized triangulation of any heart murmur.

12. A heart murmur analyzer for analyzing a heart of a patient for a heart murmur, the analyzer comprising:
a. a data interface for capturing a digital audio file of the heart;
b. a memory device for storing the digital audio file of the heart;
c. the memory device also storing computer executable instructions, the computer executable instructions comprising instructions for (i) rendering digital audio files of hearts into spectrograms, wherein the spectrograms are each a visual, colorized, three-dimensional spectrogram and the X, Y, and Z axis of the spectrogram are Time, Frequency, and Power, and (ii) an artificial intelligence model that analyzes visual, colorized, three-dimensional spectrograms of hearts;
d. a computer and processor for processing the computer executable instructions, the processing comprising (i) rendering the digital audio file into a first spectrogram, wherein the first spectrogram is a visual, colorized, three-dimensional spectrogram and the X, Y, and Z axis of the spectrogram are Time, Frequency, and Power, and (ii) inputting the first spectrogram into the artificial intelligence model trained by previously acquired and graded multiple spectrograms of the same and/or different hearts; and
e. a display for outputting information, the information comprising the first spectrogram and the analysis of the first spectrogram from the artificial intelligence model, wherein the analysis comprises an identification of any heart murmur.

13. The analyzer of claim 12, wherein the output information is provided in real-time.

14. The analyzer of claim 12, wherein the analysis further comprises an evaluation of the severity grading of any heart murmur.

15. The analyzer of claim 12, wherein the analysis further comprises an evaluation of the progression of any heart murmur.

16. The analyzer of claim 12, wherein the analysis further comprises an evaluation of the predictive estimation of heartbeat cessation.

17. The analyzer of claim 12, wherein the analysis further comprises an evaluation of the auscultatory peripheral blood flow turbulence sounds.

18. The analyzer of claim 12, wherein the analysis further comprises an evaluation of spectrograms from the same patient from one or more previous examinations of the patient.

19. The analyzer of claim 12, wherein the analysis further comprises comparisons of spectrograms from the same species as the patient with the first spectrogram from the patient.

20. The analyzer of claim 12, wherein the analysis further comprises an evaluation of spectrograms across different species compared with the first spectrogram from the patient.

21. The analyzer of claim 12, wherein the analysis comprises an evaluation of the approximate valve localization of any heart murmur.

22. The analyzer of claim 12, wherein the analysis further comprises an evaluation of the exact anatomical localized triangulation of any heart murmur.

23. A method of analyzing a heart of a patient for a heart murmur, the method comprising;
a. capturing a digital audio file of the heart;
b. rendering the digital audio file into a first spectrogram, wherein the first spectrogram is a visual, colorized, three-dimensional spectrogram and the X, Y, and Z axis of the spectrogram are Time, Frequency, and Power;
c. inputting the first spectrogram into an artificial intelligence model trained by previously acquired and graded multiple spectrograms of the same and/or different hearts; and
d. outputting the first spectrogram and an analysis of the first spectrogram by the artificial intelligence model, said analysis comprising the identification of any heart murmur; wherein the outputting of the first spectrogram and the analysis of the first spectrogram is provided in real-time and the analysis further comprises (i) an evaluation of the severity grading of any heart murmur; (ii) an evaluation of the progression of any heart murmur; (iii) an evaluation of the predictive estimation of heartbeat cessation; (iv) an evaluation of the auscultatory peripheral blood flow turbulence sounds; (v) an evaluation of multiple spectrograms from the same patient from one or more previous examinations of the patient; (vi) comparisons of multiple spectrograms from the same species as the patient with the first spectrogram from the patient; (vii) an evaluation of multiple spectrograms across different species compared with the first spectrogram from the patient; (viii) an evaluation of the approximate valve localization of any heart murmur; and/or (ix) an evaluation of the exact anatomical localized triangulation of any heart murmur.
